(19) United States
(12) Patent Application Publication
(10) Pub. No.: US 2023/0127989 A1
(43) Pub. Date: Apr. 27, 2023

(54) Wasp Feeding Station

(71) Applicant: Daradh TONER, Myshall (IE)

(72) Inventor: Daradh TONER, Myshall (IE)

(21) Appl. No.: 17/914,683

(22) PCT Filed: Mar. 29, 2021
(86) PCT No.: PCT/EP2021/058205

§ 371 Date: Sep. 26, 2022

(30) Foreign Application Priority Data
Mar. 27, 2020 (IE) S2020/0051

(51) Int. Cl. A01K 53/00

(52) U.S. Cl. CPC A01K 53/00

(57) ABSTRACT

A wasp feeding station (1) has a base frame (2) with a liquid food storage tank (3) mounted on the base frame (2) for storing liquid food for wasps. Mounted on top of the base frame (2) is a liquid food reservoir (4) which is supplied with liquid food from the storage tank (3). The storage tank (3) and the reservoir (4) have heaters which are operable to maintain the liquid food within a desired temperature range. A weather protection dome (5) is mounted on top of the reservoir (4). Access ports (8) on each side of the dome (6) allow through passage of wasps to feed at the reservoir (4).

### INTRODUCTION

This invention relates to a wasp feeding station.

### BACKGROUND OF THE INVENTION

Wasp pest infestations in summer and autumn is an annual worldwide problem resulting in loss of business revenue and creating hazard areas in locations ranging from town squares to theme parks. Vineyards, fruit farms and orchards can all suffer financially when starvation causes wasps to feast upon ripening fruit resulting in damage and disease to valuable crops and increased sting hazards for staff. Wasps become a stressor on honey bee populations during this hunger period and often destroy beehive colonies to access the food sources within. During these months, socializing, outdoor dining and events in both urban and rural locations can be hazardous, if not impossible, due to large swarms of foraging wasps. Public spaces such as parks, playgrounds and beaches can often become no-go areas due to plagues of wasps intensely searching for their next meal.

Current methods of addressing wasp infestation problems are by extermination techniques, all attempting to manage wasp infestations by killing wasps to lessen their numbers and reduce the hazards or damage caused. These current methods yield little or poor results.

Traps lure more wasps into an area often increasing the hazard potential. Trapped, distressed and dying wasps release a pheromone alerting other wasps to become defensive and aggressive and more likely to attack or sting. These traps also indiscriminately kill queen wasps, fertile males, female workers and many other beneficial insect species.

Although existing wasp pest control devices offer both liquid and solid food sources, these foods are used for luring or baiting purposes to enable the extermination of wasps by, poisoning, drowning, gluing, trapping, de-hydrating etc.

Generally, housings are configured to trap and kill wasps. Otherwise devices utilizing housings or enclosures that do not exterminate or trap at point, provide wasps with a protein based toxic food that is then returned to hive and fed to the young larva to poison the entire colony. This last method is employed mainly were the species is considered invasive.

The use of sprays, poisons and insecticides to kill wasps have unfortunate environmental repercussions. These chemicals are highly toxic and cause long-term damage to soil and pollute ground water. As a consequence of cross contamination and bio amplification these toxins cause harm to many other wild life species, while also tainting the food supply and causing a range of potential health issues for humans. Many of these chemical insecticides are now being restricted or banned.

Social wasps are highly evolved insects and display an advanced level of social organisation—eusocial. Wasps are an integral link of the food web and play a vital role maintaining harmony in our ecosystems. A healthy native wasp population can be noted as a ‘good barometer’ as to the overall health of local biodiversity.

As apex predators, wasps are an essential insect in world food production. They spend the most part of their life cycle as natural bio control agents hunting lepidopteran pests that would otherwise destroy entire food crops. One estimate from the UK states that wasps are responsible for removing 14,000 tonnes of unwanted pests from fruit, crops and gardens annually.

While foraging for insects or nectar wasps are active pollinators collecting pollen on their almost invisible hairs inadvertently transferring it from flower to flower.

Hibernating queen wasps store essential yeasts in their gut overwinter, specifically yeasts of the species, Saccharomyces cerevisiae. Keeping these alive in her body as she sleeps through winter, the following season she passes these yeasts on to her colony who go out and disperse them amongst the crops that produce wine, beer and bread.

Wasps are also a food source for many other creatures such as birds, spiders, dragonflies, reptiles, frogs, toads and a variety of small mammals which all feed on wasps as part of their diet.

A small number of non-stinging male drone wasps are born at seasons end for reproductive purposes otherwise all adult wasps are female have a proboscis and cannot consume solid foods. These adult wasps spend their lives hunting protein rich insect's and insect larvae which they then return to the hive and feed in a masticated form to the young wasp larvae who in return secrete a nutritional syrup from glands that the adults then consume, a food exchange process known as trophallaxis. Aside from nectar and sap this hive larvae produced syrup is the adult wasps primary food source.

This food exchange process begins to naturally decline in late summer as the hive queen begins to lay ever decreasing quantities of eggs, resulting in increasing populations of adult wasps with no larvae to feed and therefore no liquid food to consume.

With an ever depleting food supply and facing starvation wasps have little choice but resort to seeking out alternative food sources and so commences the annual the wasp pest season when wasps swarm and infest public and private areas, businesses, industries and ripened fruits seeking nourishment.

This annual natural phenomenon causing the dietary shift of wasps from beneficial insect predator to food scavenger has unfortunately earned the wasp its pest reputation amongst society.

Due to a wide range of contributing factors, world biodiversity loss and insect decline is now at crisis point. We have a collective responsibility to create and implement dynamic solutions to protect and restore our ecosystems while at the same time solve our perceived problems. An objective of the wasp feeding station of this invention is to produce wasp pest free environments, Accomplishing this objective will result in benefit to businesses, organisations, agriculture and society and achieving this without causing harm to the ecosystem will have a positive impact on biodiversity and the environment.

For convenience throughout this patent specification, we refer to a wasp feeding station, however, this term as used herein should be taken to include feeding stations for other flying insects such as bees, butterflies, moths and the like, and references to wasps generally herein should be taken to include these other flying insects also.

### SUMMARY OF THE INVENTION

According to the invention there is provided wasp feeding station comprising:
a liquid food reservoir;
a liquid food storage tank connected to the reservoir for delivery of liquid food from the liquid food storage tank to the reservoir;
the liquid food storage tank being connected to the liquid food reservoir by a liquid food supply system which is operable to deliver liquid food from the liquid food storage tank to the liquid food reservoir to maintain a constant supply of liquid food in the liquid food reservoir.

In one embodiment of the invention the reservoir comprises at least one feeding channel having an access opening sufficiently narrow to prevent a wasp falling into the feeding channel.

In another embodiment a landing pad is provided adjacent the access opening of the feeding channel.

In another embodiment the landing pads are provided at opposite sides of the feeding channel.

In another embodiment the reservoir is mounted within a weather protection enclosure having at least one access port to allow through passage of wasps to feed at the reservoir and to exit the weather protection enclosure.

In another embodiment the wasp feeding station further includes a temperature control system for regulating the temperature of the liquid food.

In another embodiment the temperature control system has heating apparatus and/or cooling apparatus for maintaining the liquid food within a desired temperature range.

In another embodiment a sterilizer is mounted within the liquid food storage tank for sanitising liquid food therein.

In another embodiment the sterilizer comprises a UV light source.

In another embodiment an additive dosing tank is connected to the liquid food storage tank for delivery of additives into the liquid food.

In another embodiment the reservoir comprises a feeding pool having a base with upstanding side walls, a feeding plate mounted within the pool, the feeding plate having a plurality of feeding channels for reception of liquid food.

In another embodiment a top surface of the feeding plate forms landing pads at opposite side of the feeding channels.

In another embodiment one or more thermal pads are mounted on the base of the reservoir, a thermostat associated with the thermal pads being operable to control operation of the thermal pads to maintain the temperature of liquid food in the reservoir within a desired temperature range by heating or cooling the liquid food in the reservoir.

In another embodiment a scraper blade extends between opposite side walls and engages a top surface of the feeding plate, the scraper blade having a drive assembly for movement of the scraper blade across the top surface.

In another embodiment the reservoir has a level adjuster.

In another embodiment the reservoir has height adjustable feet forming the level adjuster for mounting the reservoir on a support and levelling the reservoir on the support.

In another embodiment the reservoir is supported on suspension arms which are length adjustable to provided the level adjuster for the reservoir.

In another embodiment the reservoir comprises a plurality of vertically stacked feed troughs.

In another embodiment the feed troughs are connected by complementary interengagable formations on a top and on a bottom of each feeding trough.

In another embodiment the complementary interengagable formations comprise associated male and female connector elements.

In another embodiment when the liquid food storage tank has a mixer for mixing liquid food stored in the storage tank.

In another embodiment the mixer comprises a mixer pump mounted in a recirculating pipeline, the mixer pump having a mixer pump inlet connected to a storage tank outlet and a mixer pump outlet connected to a storage tank inlet.

In another embodiment a feed transfer pump has a feed transfer pump inlet connected to an outlet of the storage tank and a feed transfer pump outlet connected to an inlet of the reservoir.

In another embodiment the weather protection enclosure has a plurality of spaced-apart landing pads are mounted at each access port.

In another embodiment an infra-red counting gate is mounted at each access port.

In another embodiment the weather protection enclosure comprises a domed cover mounted on top of the reservoir.

In another embodiment the weather protection enclosure comprises an upstanding housing within which the reservoir and storage tank are mounted, the reservoir being mounted at a top of the housing.

In another embodiment the storage tank is mounted on a base framework having height adjustable feet.

In another embodiment the wasp feeding station includes monitoring and communication equipment to enable remote monitoring of the wasp feeding station.

In another embodiment the monitoring and communication equipment are mounted on a tower or on the housing.

In another embodiment the monitoring and communication equipment includes one or more of cameras, lights, weather station, wireless connectivity and GPS.

In another embodiment a filter is mounted in a liquid food return line communicating between the reservoir and the liquid food storage tank.

In another embodiment an additive dosing tank is provided having an outlet communicating with the liquid food storage tank for delivering additives to liquid food in the liquid food storage tank.

### BRIEF DESCRIPTION OF THE DRAWINGS

The invention will be more clearly understood from the following description of some embodiments thereof, given by way of example only, with reference to the accompanying drawings, in which:

FIG. 1 is a perspective view of a wasp feeding station according to the invention;

FIG. 2 is an exploded perspective view of the wasp feeding station;

FIG. 3 is a detail perspective view showing a base frame and tower portion of the wasp feeding station;

FIG. 4 is a detail exploded perspective view of the base frame and tower portion of the wasp feeding station;

FIG. 5 is a detail perspective view of a reservoir forming portion of the wasp feeding station;

FIG. 6 is a detail exploded perspective view of the reservoir;

FIG. 7 is an underneath perspective view of the reservoir;

FIG. 8 is a detail perspective view showing portion of the reservoir;

FIG. 9 is a detail perspective view showing a feeding plate forming portion of the reservoir assembly;

FIG. 10 is an enlarged detail perspective view showing portion of the feeding plate;

FIG. 11 is an enlarged detail perspective cut-away view showing portion of the reservoir assembly;

FIG. 12 is a detail perspective view showing a weather protection dome portion of the wasp feeding station;

FIG. 13 is a detail exploded perspective view of the weather protection dome portion;

FIG. 14 is a perspective view of another wasp feeding station according to a second embodiment of the invention;

FIG. 15 is a partially cut-away perspective view of the wasp feeding station shown in FIG. 14;

FIG. 16 is an enlarged detail view showing a reservoir portion of the wasp feeding station of FIG. 14;

FIG. 17 is an enlarged detail perspective view showing a base frame portion of the wasp feeding station of FIG. 14;

FIG. 18 is a detail plan view showing portion of the wasp feeding station of FIG. 14;

FIG. 19 is a detail partially exploded perspective view of a part of the reservoir portion of the wasp feeding station of FIG. 14;

FIG. 20 is an enlarged detail partially exploded perspective view showing an outlet of the reservoir portion;

FIG. 21 is an enlarged detail perspective view of the reservoir part shown in FIG. 19;

FIG. 22 is a detail end elevational view of the reservoir part shown in FIG. 19;

FIG. 23 is a detail perspective view of a feeding trough portion of the reservoir;

FIG. 24 is a detail partially cut-away perspective view showing portion of the feeding trough;

FIG. 25 is an enlarged detail perspective view showing an end portion of the feeding trough;

FIG. 26 is a detail sectional view showing portion of the feeding trough;

FIG. 27 is a detail partially exploded sectional plan view of portion of the feeding trough;

FIG. 28 is a detail perspective view showing a header support tube portion of the reservoir;

FIG. 29 is a detail partially sectioned perspective view of the header support tube portion of FIG. 28;

FIG. 30 is an enlarged detail perspective view showing a C-link pipe of the reservoir;

FIG. 31 is an enlarged detail perspective view showing part of the C-link pipe;

FIG. 32 is a detail elevational view showing a feed liquid distribution manifold portion of the wasp feeding station;

FIG. 33 is a detail partially sectioned plan view of the feed liquid distribution manifold;

FIG. 34 is a detail perspective view showing portion of the feed liquid distribution manifold;

FIG. 35 is a perspective view of another reservoir assembly for a wasp feeding station of the invention;

FIG. 36 is a detail partially cut-away perspective view of the reservoir assembly shown in FIG. 35;

FIG. 37 is an exploded perspective view of the reservoir assembly shown in FIG. 35;

FIG. 38 is a perspective sectional view of portion of the reservoir assembly shown in FIG. 35;

FIG. 39 is a perspective sectional view of portion of the reservoir assembly shown in FIG. 35;

FIG. 40 is a perspective view of a wasp feeding station according to another embodiment of the invention;

FIG. 41 is an exploded perspective view of the wasp feeding station of FIG. 40;

FIG. 42 is a detail perspective view showing a base frame portion of the wasp feeding station of FIG. 40;

FIG. 43 is a detail exploded perspective view of the base frame of the wasp feeding station of FIG. 40; and

FIG. 44 is a detail underneath perspective view of a reservoir portion of the wasp feeding station of FIG. 40.

### DETAILED DESCRIPTION OF HE PREFERRED EMBODIMENTS

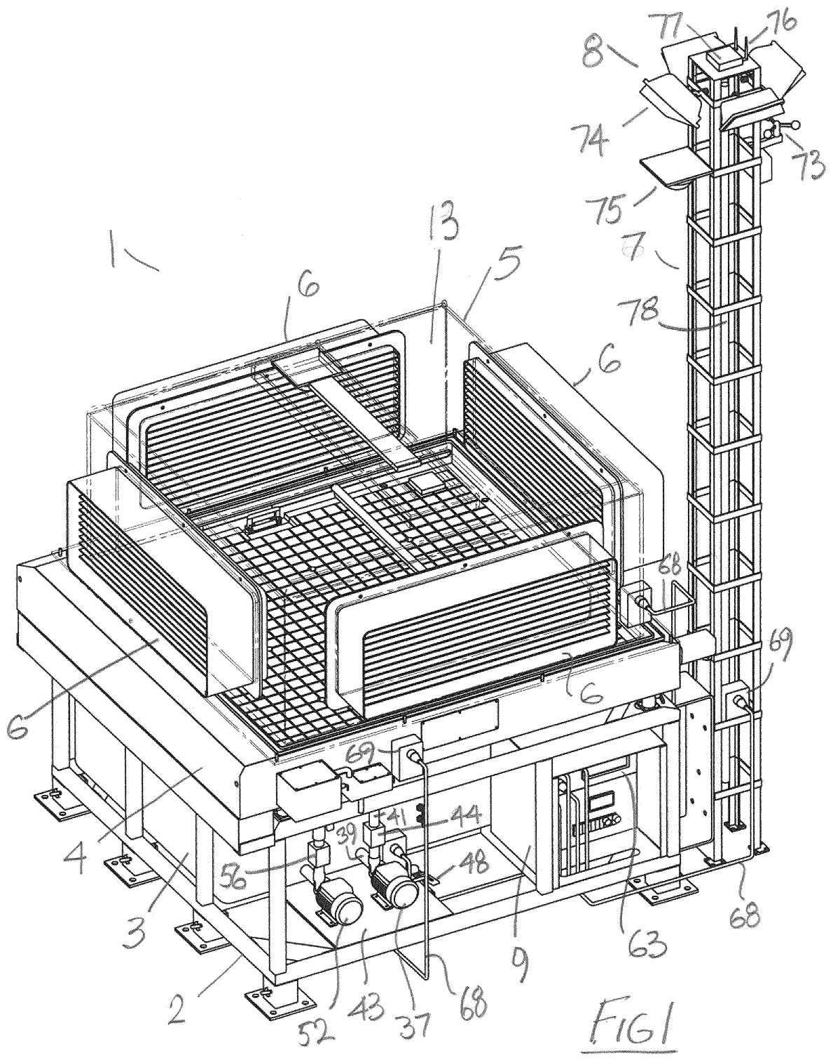

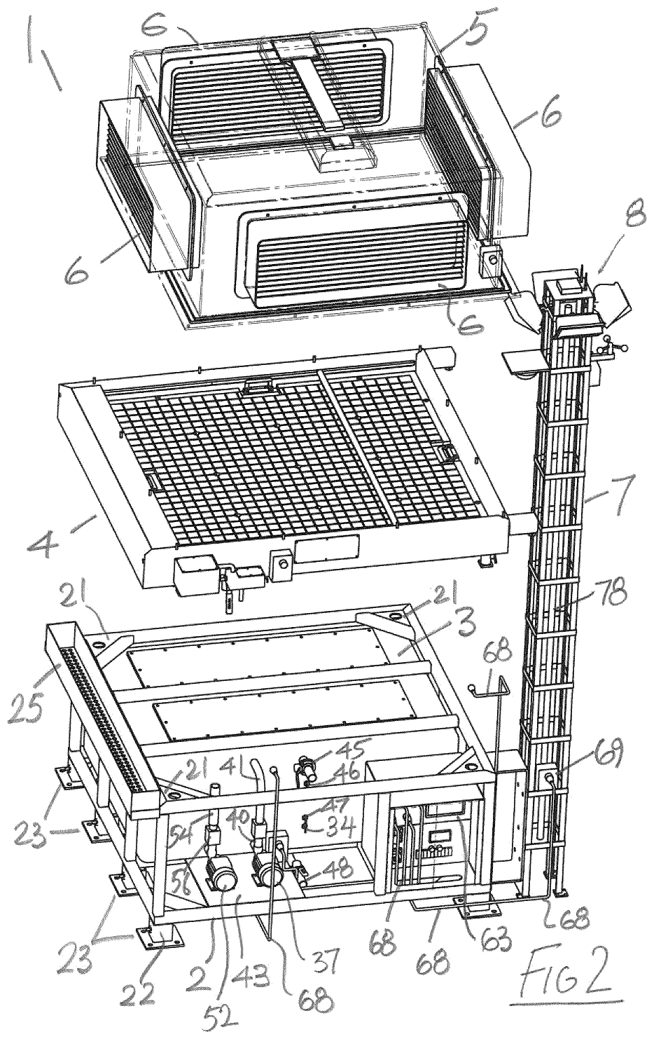

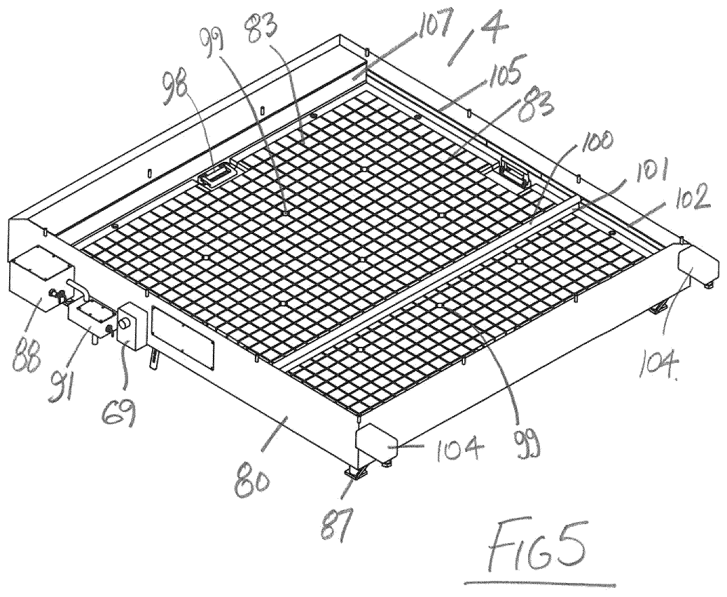

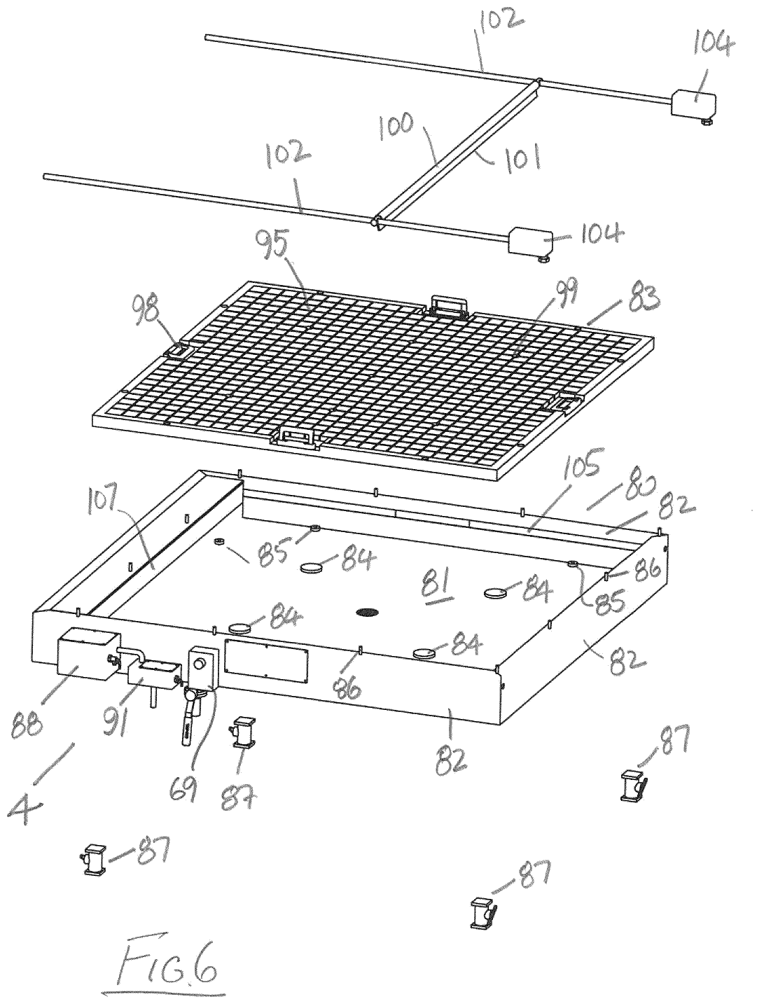

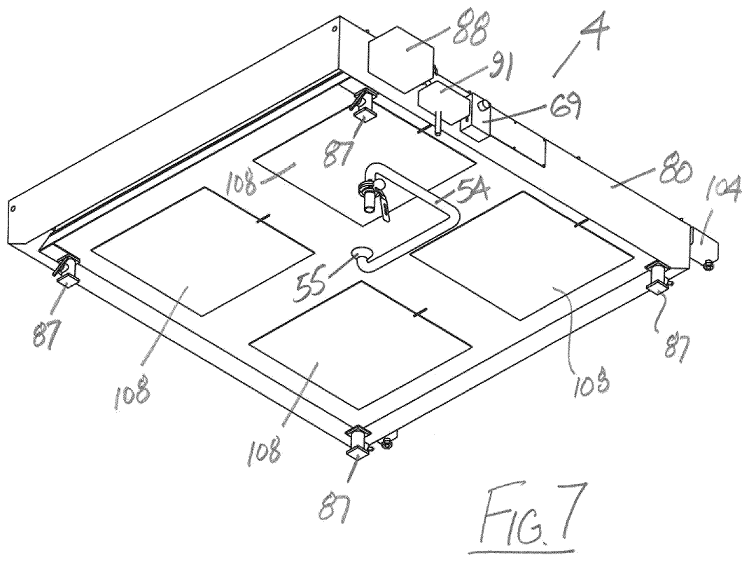

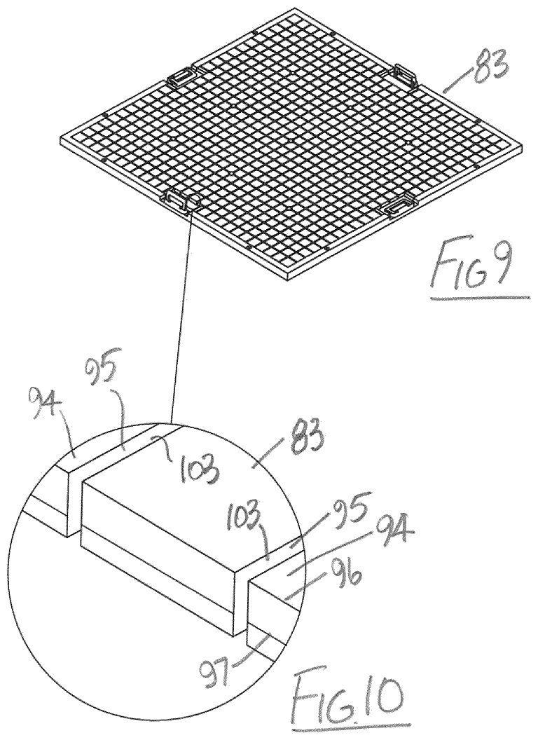

Referring to the drawings, and initially to FIG. 1 to FIG. 13 thereof, there is illustrated a wasp feeding station according to the invention indicated generally by the reference numeral 1. The wasp feeding station 1 has a base frame 2. A liquid food storage tank 3 is mounted on the base frame 2 for storing liquid food for wasps. Mounted on top of the base frame 2 is a liquid food reservoir 4 forming a liquid food pool which is supplied with liquid food from the storage tank 3 as will be described in more detail later. Heaters in the storage tank 3 are occasionally operable as required to maintain homogenous suspension of liquid food. Heaters on a base of the reservoir 4 are initially operable to produce airborne aroma. A weather protection dome 5 is mounted on top of the reservoir 4 to define a feeding compartment 13. Access ports 6 on each side of the dome 5 allow through passage of wasps into the feeding compartment 13 to feed at the reservoir 4. An upstanding tower 7 at one side of the base frame 2 supports monitoring and communications equipment indicated generally by the reference numeral 8. A weatherproof control cabinet 9 mounted on the base frame 2 houses power supply, control equipment and multipin connectors 69 to connect the components of the wasp feeding station 1 via interconnect cables 68 allowing for operation of the wasp feeding station 1.

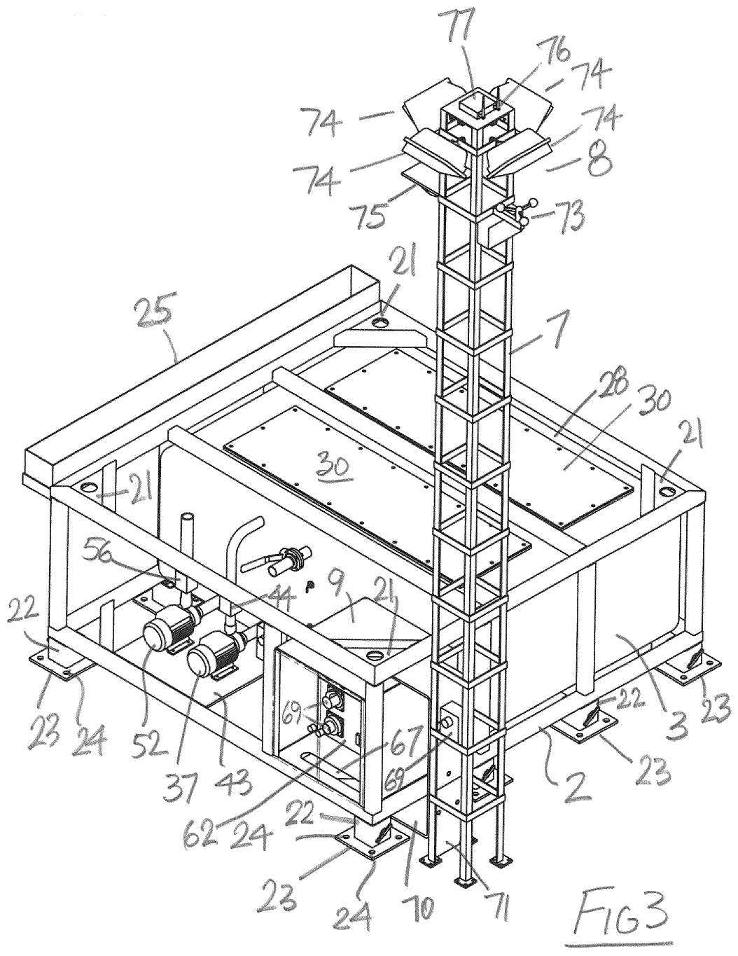

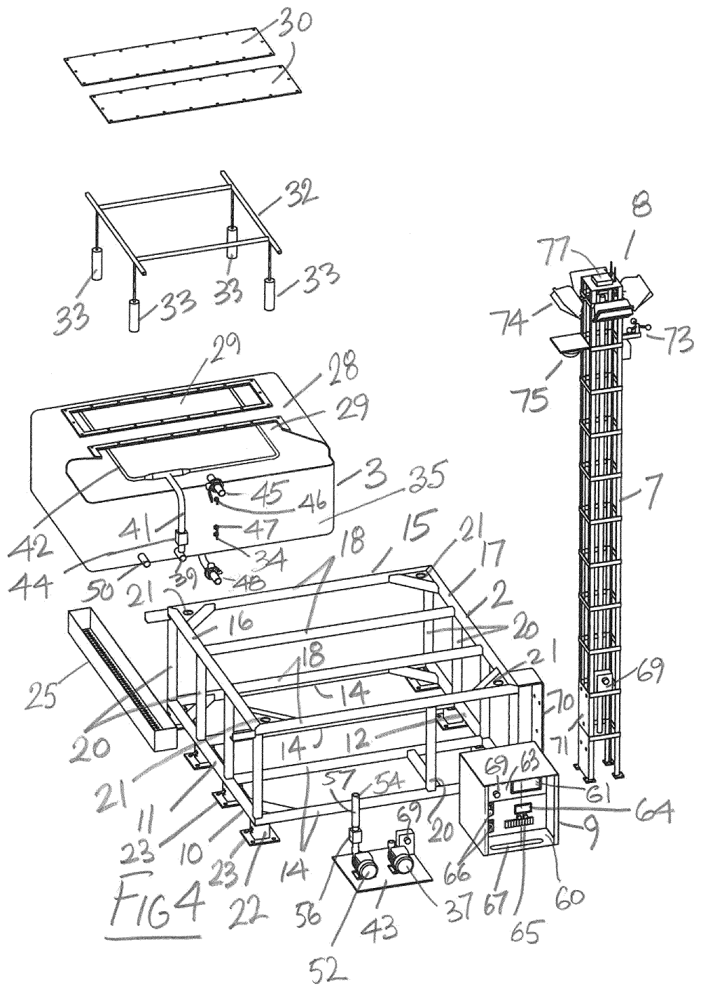

Referring in particular to FIG. 3 and FIG. 4, the base frame 2 has a rectangular bottom frame element 10 comprising bottom side rails 11, 12 interconnected by a number of spaced-apart bottom crossbars 14 extending therebetween. Similarly, the base frame 2 has a rectangular top frame element 15 comprising top side rails 16, 17 interconnected by a number of spaced-apart top crossbars 18 extending therebetween. A number of spaced-apart upstanding posts 20 extend between the bottom frame element 10 and the top frame element 15 and support the top frame element 15 parallel to and spaced above the bottom frame element 10.

Gusset plates 21 are mounted inside each corner of the top frame element 15 upon which the reservoir 4 is supported. At a bottom of the base frame 2 a number of spaced-apart height adjustable legs 22 project downwardly from each bottom side rail 11, 12.

Each leg 22 is located beneath a post 20 and has a flanged foot pad 23 with through fixing holes 24. A disposal tray 25 associated with the reservoir 4 is mounted along one side of the top frame element 15.

The liquid food storage tank 3 is mounted within the base frame 2. A top wall 28 of the storage tank 3 has access openings 29 closed by demountable tank inspection hatches 30. A carrier frame 32 supports a number of heating elements 33 within the storage tank 3. A thermostat 34 associated with the heating elements 33 is mounted on a side wall 35 of the storage tank 3 and is operable to control operation of the heating elements 33 to maintain liquid food in the storage tank 3 within a desired temperature range.

A mixer is provided for mixing liquid food to maintain homogenous suspension of liquid food stored in the storage tank 3. In this case the mixer is a mixer pump 37 having a mixer pump inlet connected to an outlet 39 of the storage tank 3 and a mixer pump outlet 40 connected by a feed pipe 41 to irrigation pipework 42 within the storage tank 3 at a top of the storage tank 3. The mixer pump 37 is mounted on a pump mounting plate 43 on the bottom frame element 10 of the base frame 2. Liquid food mix is drawn through the mixer pump 37 and returned to the storage tank 3 at a higher pressure through the irrigation pipework 42. A flowmeter 44 is mounted in the feed pipe 41.

The storage tank 3 can be filled through a main inlet valve 45 at a top of the storage tank 3. A high liquid level sensor 46 and a low liquid level sensor 47 are mounted on the side wall 35 of the storage tank 3. When the liquid food within the storage tank 3 drops to the low level sensor 47 the storage tank 3 is re-filled up to the high liquid level sensor 46. A main outlet valve 48 at a bottom of the storage tank 3 facilitates emptying of the storage tank 3.

An outlet 50 at a bottom of the storage tank 3 connects to an inlet of a feeder pump 52 mounted on the pump mounting plate 43. An outlet of the feeder pump 52 connects through a reservoir supply line 54 with an inlet 55 of the reservoir 4 via a flexible hose 57. A flowmeter 56 is mounted in the reservoir supply line 54.

The control cabinet 9 is mounted on the base frame 2 and has lockable service doors 60. Mounted within the control cabinet 9 is a technician interface computer screen 61, a power supply unit—P.S.U. 62, multiprocessor 63, multipin connectors 69, backup battery and indicator 64, A.C. protection isolators 65 and A.C. input/output connectors 66. A cable access hatch 67 is provided by an opening in a floor of the control cabinet 9. Interconnect cables 68 connect between junction boxes with input multipin connectors 69 at the various components to provide power and communication.

A tower connector plate 70 is mounted at one side of the base frame 2 and is connected to an associated connector panel 71 at a bottom of the tower 7. At a top of the tower 7 the monitoring and communications equipment 8 includes a weather station 73, external lights 74, an external camera 75, GPS module 76 and wireless communication module 77. This allows communication with a remote monitoring station in order to remotely monitor and control operation of the wasp feeding station 1. Fixed within the tower 7 is a system status indicator light 78.

Referring in particular to FIG. 5 to FIG. 11, the reservoir 4 forms a wasp feeding pool and comprises a rigid box framework 80 having a rectangular base 81 with upstanding peripheral side walls 82. A feeding plate 83 is mounted within the reservoir box framework 80 supported on risers 84 on the base 81 and secured to plate fixings 85 on the base 81. Upstanding peg fixings 86 at a top of the side walls 82 engage and secure the dome 5 on top of the reservoir 4. Adjustable coupler feet 87 on an underside of the reservoir 4 at each corner attach securely to the gusset plates 21 at the top of the base frame 2. These adjustable coupler feet 87 facilitate fine levelling of the reservoir 4.

A wet float chamber 88 is mounted on a side wall 82 of the reservoir 4 and communicates through interconnecting opening 79 with an interior of the reservoir 4. A float switch 89 within the wet float chamber 88 controls filling of the reservoir 4 with liquid food by switching the feeder pump 52 on and off.

Also mounted within the wet float chamber 88 is a thermostat 90 which together with associated heating pads 108 on an underside of the reservoir 4 initially heat liquid food within the reservoir 4, creating evaporation and thus releasing airborne aroma of the liquid food into the atmosphere to attract wasps to the wasp feeding station 1.

An overflow chamber 91 on the side wall 82 of the reservoir 4 is connected to the wet float chamber 88 by an overflow pipe 92. A sensor 93 within the overflow chamber 91 monitors pump malfunction. Removable service covers on top of each chamber 88, 91 allow access to both chambers 88, 91 if required.

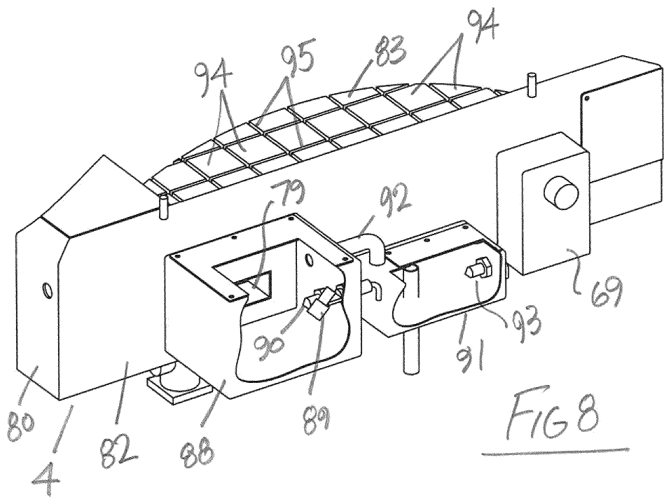

The feeding plate 83 provides a surface area from which the wasps feed and comprises a network or grid of feeding channels formed by canals 95 with through holes 99 between which landing and crawl spaces 94 are situated. FIG. 10 indicates a canal high level 96 and canal low level 97 corresponding to when the feeder pump 52 is switched off and on respectively to maintain the level of liquid food in the reservoir 4 and in the canals 95 of the feeding plate 83, Flush mounted lifting handles 98 are provided on the feeding plate 83. It will be noted that each canal 95 has an upwardly open access opening 103 which is sufficiently narrow to prevent a wasp falling into the canal whilst at the same time being wide enough to allow the wasp to feed at the canal 95.

A rigid cleaning arm 100 with a rubber scoop 101 or blade is mounted above the feeding plate 83 with the rubber scoop 101 engaging a top surface of the feeding plate 83. Opposite ends of the cleaning arm 100 engage associated mechanical gearing 102 located in openings 105 in opposed side walls 82 and drivably connected to reversable drive motors 104 mounted on an outer face of a side wall 82 at one end of the reservoir 4. Operation of the drive motors 104 in tandem moves the cleaning arm 100 and scoop 101 over the upper surface of the feeding plate 83 to deliver debris to a hatch 107 leading to a chute 106 which discharges the debris into the disposal tray 25. A perforated base of the disposal tray 25 retains debris while allowing excess liquid to run off.

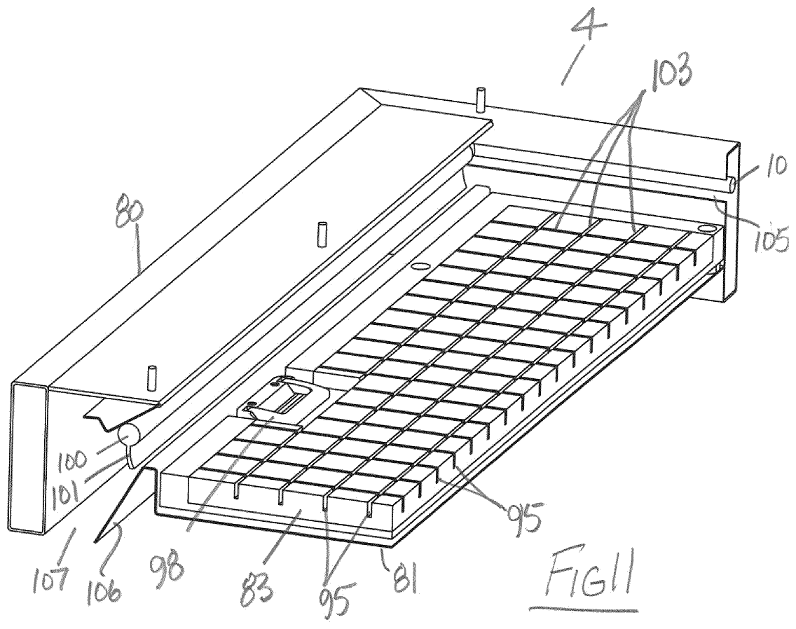

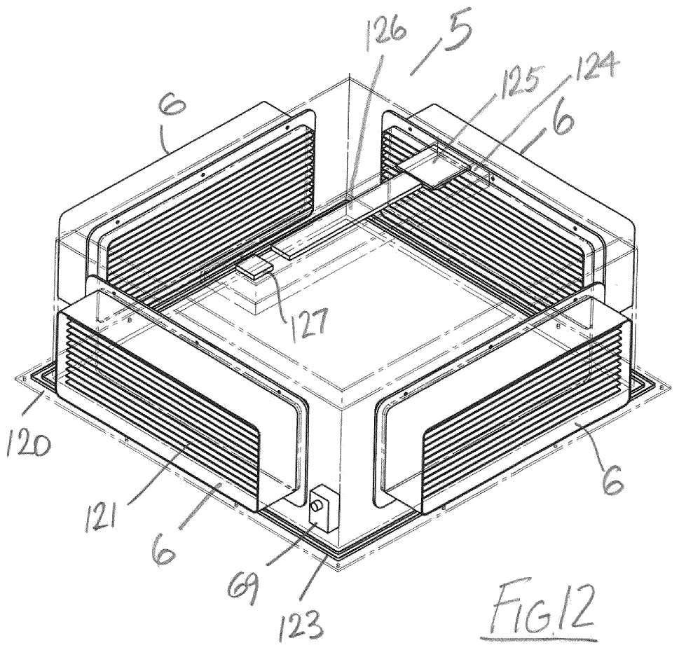

Referring in particular to FIG. 12 and FIG. 13, the dome 5 forming the weather protection enclosure has a peripheral base flange 120 with holes to receive the dome fixing pegs 86 when the flange 120 sits on top of the reservoir side walls 82. A system status indicator light 123 sits on and extends around a top face of the flange 120.

Each access port 6 has a stack of vertically spaced-apart horizontal through landing pads 121 onto which wasps land and take off from. Associated with each landing pad 121 is a vertical infra-red gate 122 through which wasps must pass to access the liquid food at the reservoir 4 within the feeding compartment 13. These infra-red gates 122 detect and count the number of wasp visits.

The dome 5 has a top opening to which is fitted a raised service compartment 124 which contains an internal camera 125, internal lighting 126 and a thermostat 127. This also provides internal mounting points for scientific instruments for research purposes. The internal lighting 126 may comprise a UV sterilization light which is operable to sanitize the liquid food.

In use, operation of the mixer pump 37 draws liquid food from the liquid food storage tank 3 and returns the liquid food to the liquid food storage tank 3 for maintaining a homogenous mix of liquid food in the liquid food storage tank 3. The feeder pump 52 operates in response to the float switch 90 in the wet float chamber 88 to deliver liquid food from the liquid food storage tank 3 to the reservoir 4 when the canal low level 97 is sensed, raising the level of liquid food in the reservoir up to the canal high level 96, thus maintaining a continuous supply of liquid food in the reservoir 4 at all times. The various heaters operate as and when required in response to the associated thermostats to maintain the liquid food within a desired temperature range. Continuous automatic operation of the wasp feeding station 1 is controlled by the multiprocessor 63. The monitoring and communications equipment 8 allows remote monitoring of each wasp feeding station 1 from a remote monitoring station from which can be directed any maintenance required and replenishment of liquid food in the liquid food storage tank 3 as required in response to consumption of the liquid food.

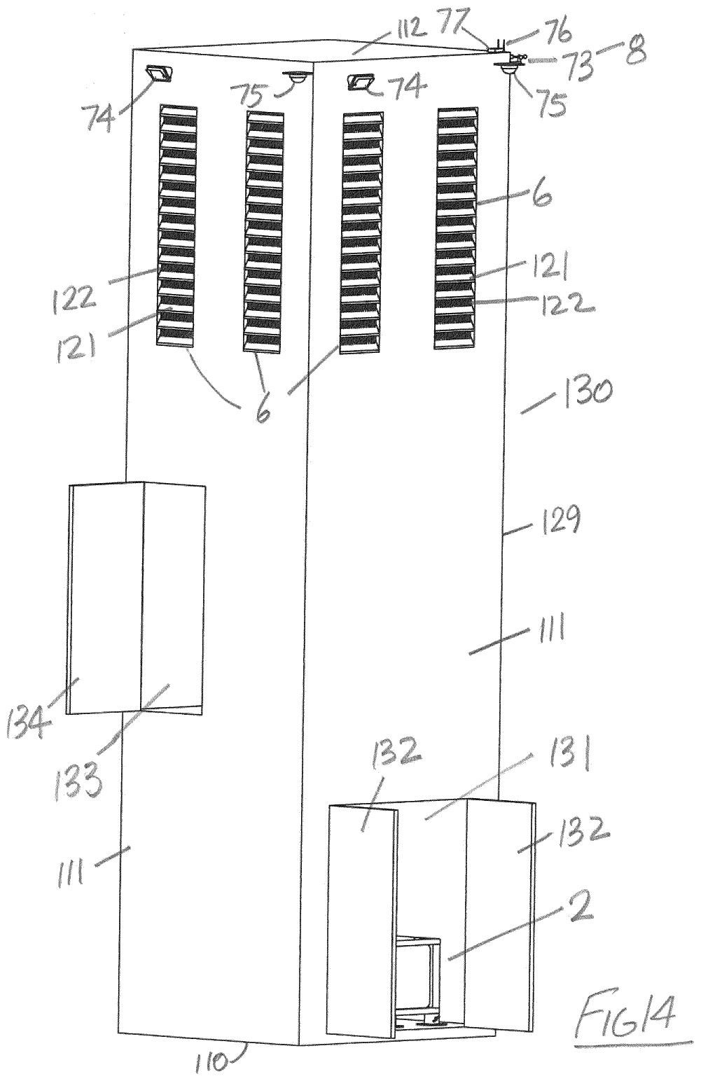

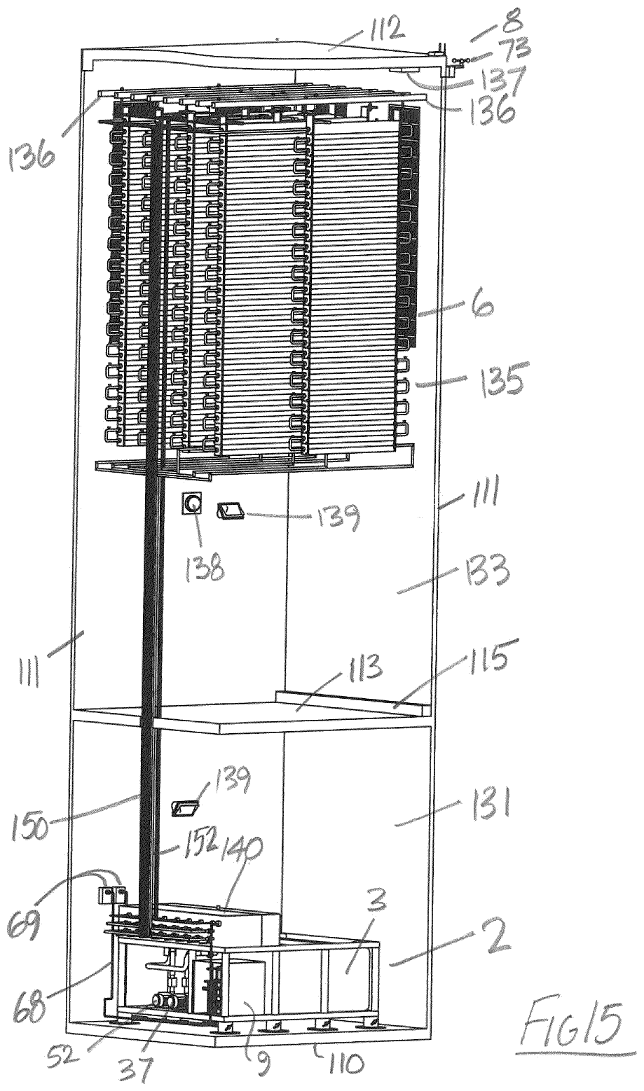

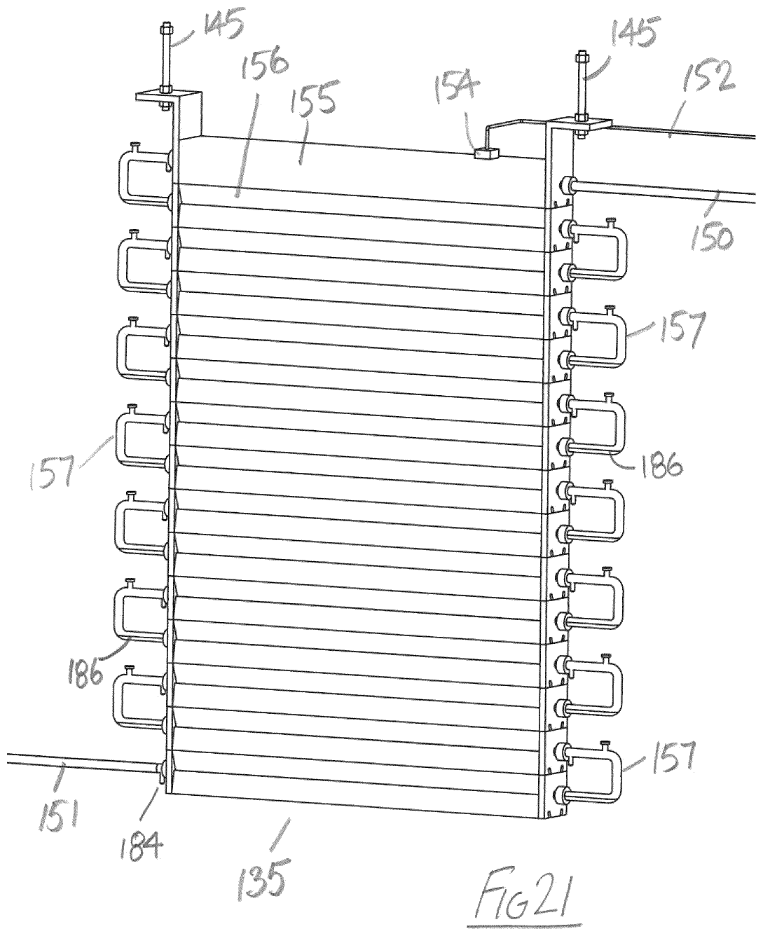

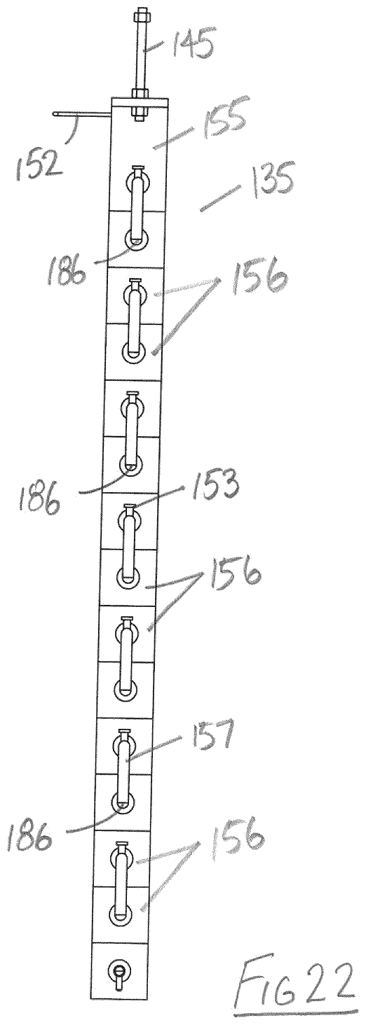

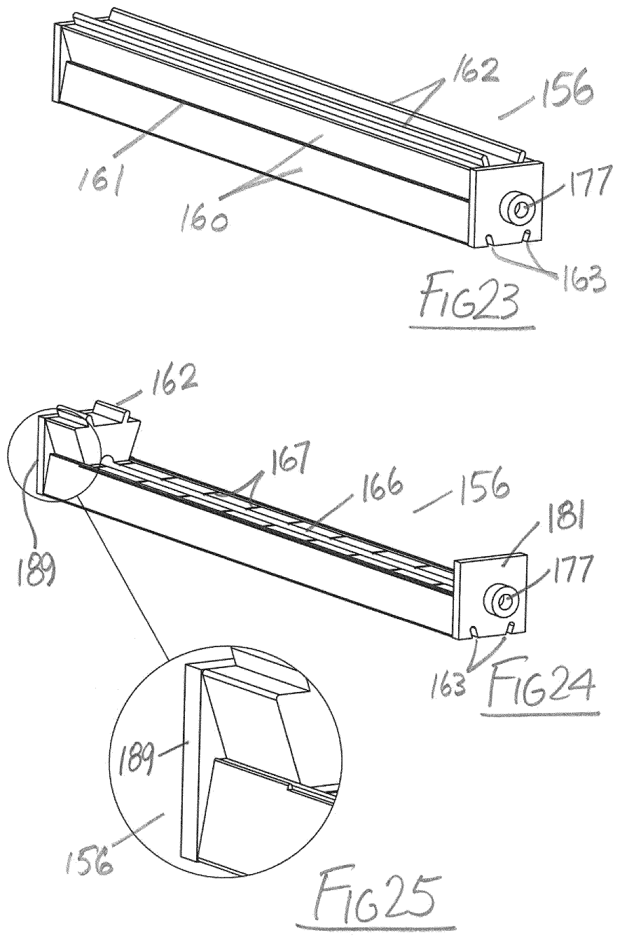

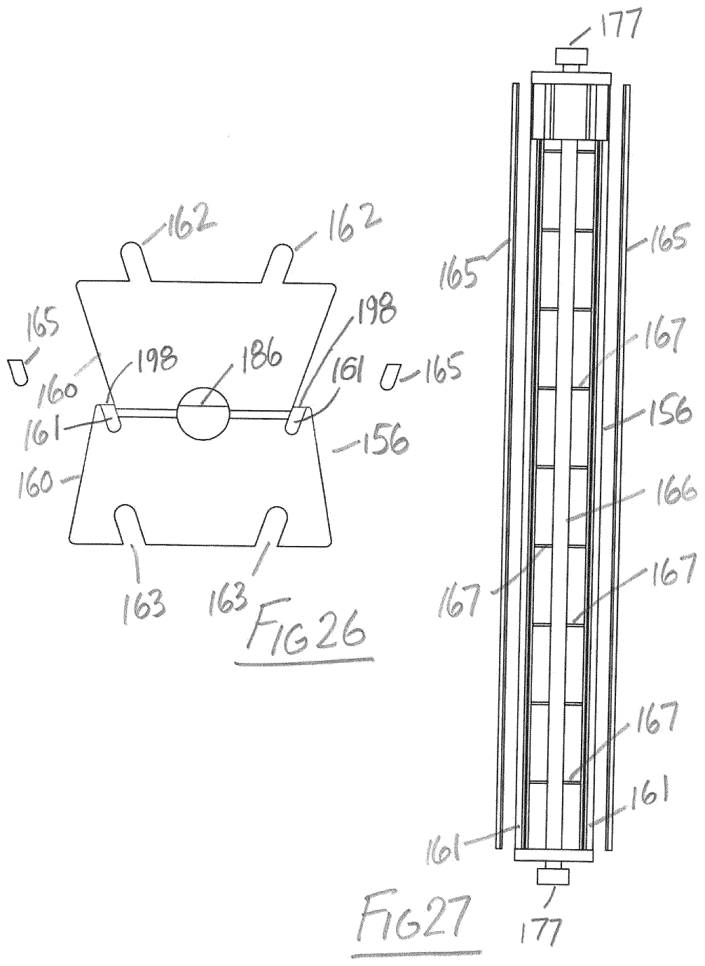

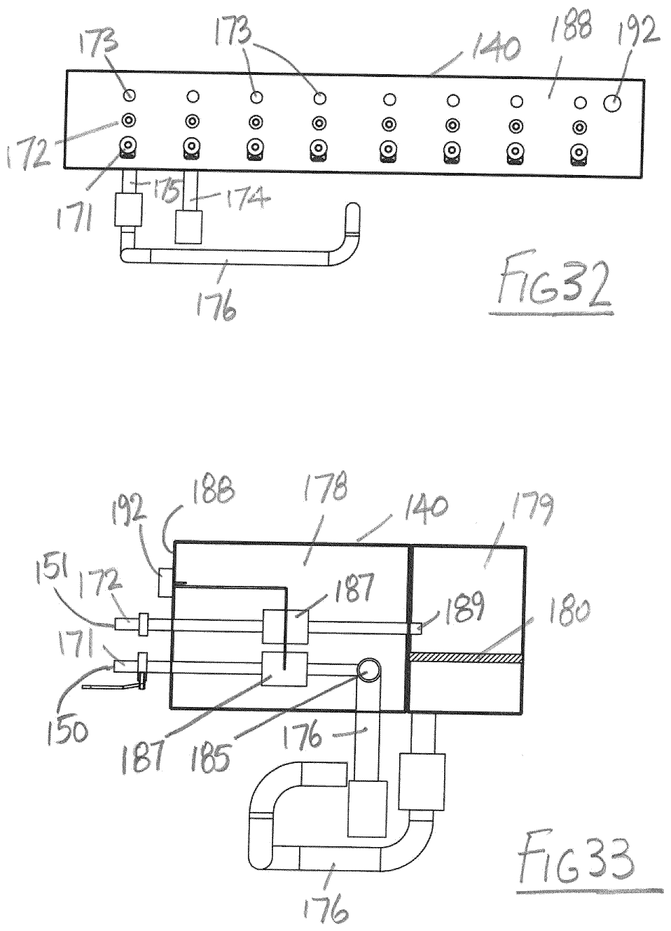

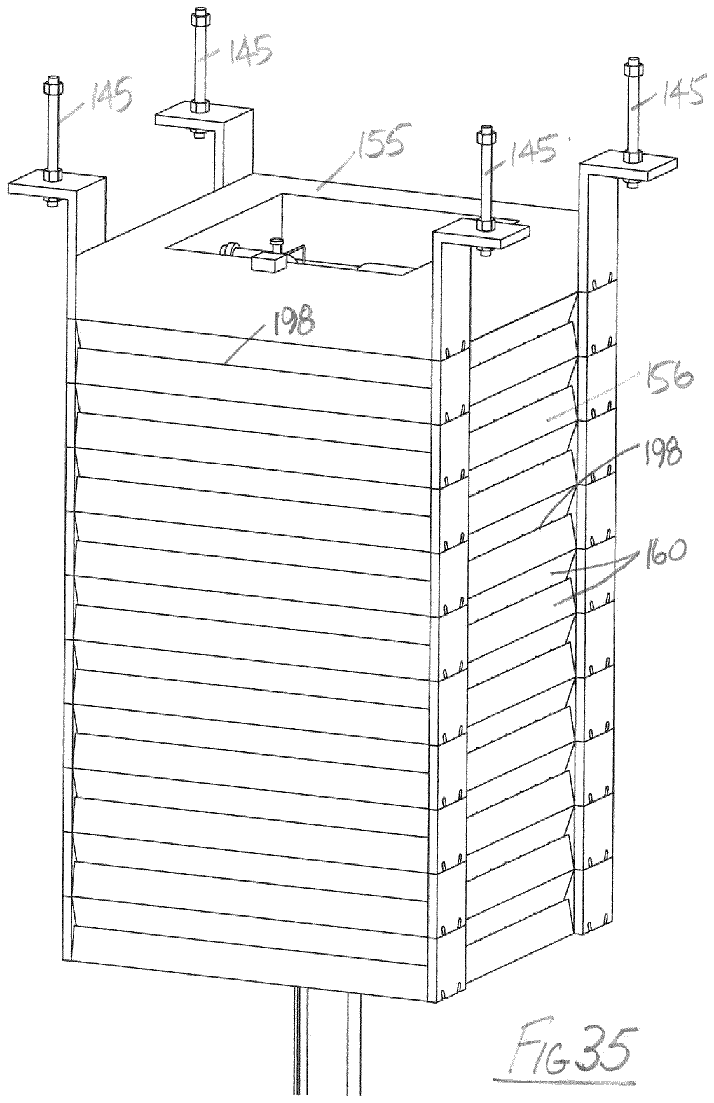

Referring now to FIG. 14 to FIG. 33, there is shown another wasp feeding station according to a second embodiment of the invention, indicated generally by the reference numeral 130. Parts similar to those described previously are assigned the same reference numerals. In this case the wasp feeding station 130 has a weather protection enclosure formed by an upstanding housing 129 having a rectangular base 110 with upstanding side walls 111 and a rectangular top wall 112 parallel to the base 110. The housing 129 has a lower compartment 131 and an upper feeding compartment 133 within which the base frame 2 with storage tank 3 and a reservoir 135 are mounted respectively, each compartment 131, 133 separated by an intermediate wall 113 which is parallel to the base 110 and the top wall 112, and each compartment 131, 133 being provided with access doors 132, 134. Power and communication is supplied to components—lighting, cameras, weather station, infrared gates, etc. by interconnect cables 68 to multipin connectors 69 mounted on an inner face of the side wall 111 of the lower compartment 131.

Referring in particular to FIG. 14 to FIG. 16 the monitoring and communications equipment 8 is in this case mounted at a top of the housing 129. Infrared counting gates 122 are mounted in access openings forming the ports 6 at a top of each side wall 111 of the housing 129 together with associated landing pads 121. A steel support beam 136 suspends the reservoir 135 on level adjusting brackets 145 at a top of the feeding compartment 133. A thermostat 137 is mounted at the top of the feeding compartment 133. Below the reservoir 135 within the feeding compartment 133 on an inner side wall of the feeding compartment 133 are mounted an internal camera 138 and lighting 139 to monitor the feeding compartment 133.

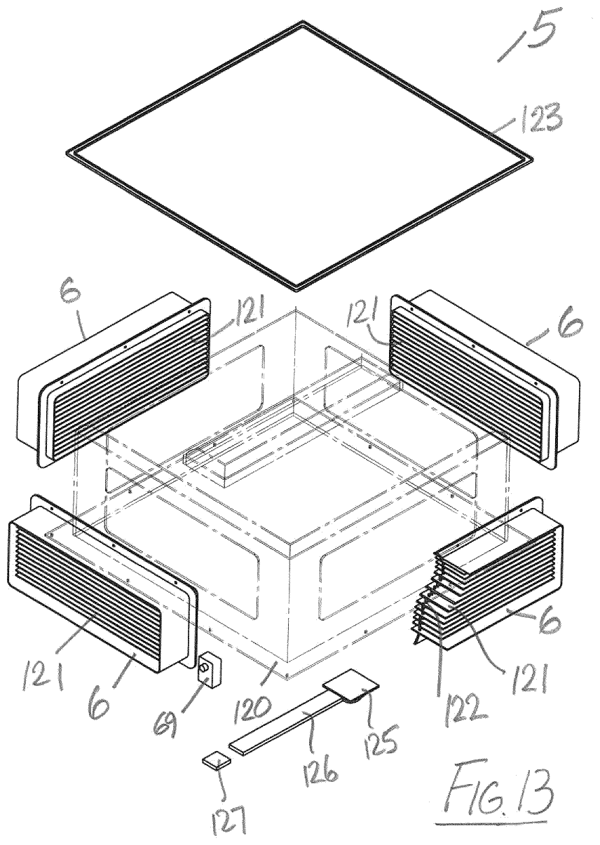

A vacuum motorized cleaning arm 115 maintains a clean floor surface 114 of the feeding compartment 133. The cleaning arm 115 is shown schematically in FIG. 15 and FIG. 18 and has an associated drive (not shown) to move the cleaning arm 115 across and back over the floor surface 114 between opposite side walls 111 to remove any debris on the floor surface 114. The cleaning arm 115 is actuated as required, typically daily.

In the lower compartment 131 a liquid distribution manifold 140 is mounted on top of the base frame 2. Flow pipework 150 and sterilization light cables 152 connect between the manifold 140 and the reservoir 135.

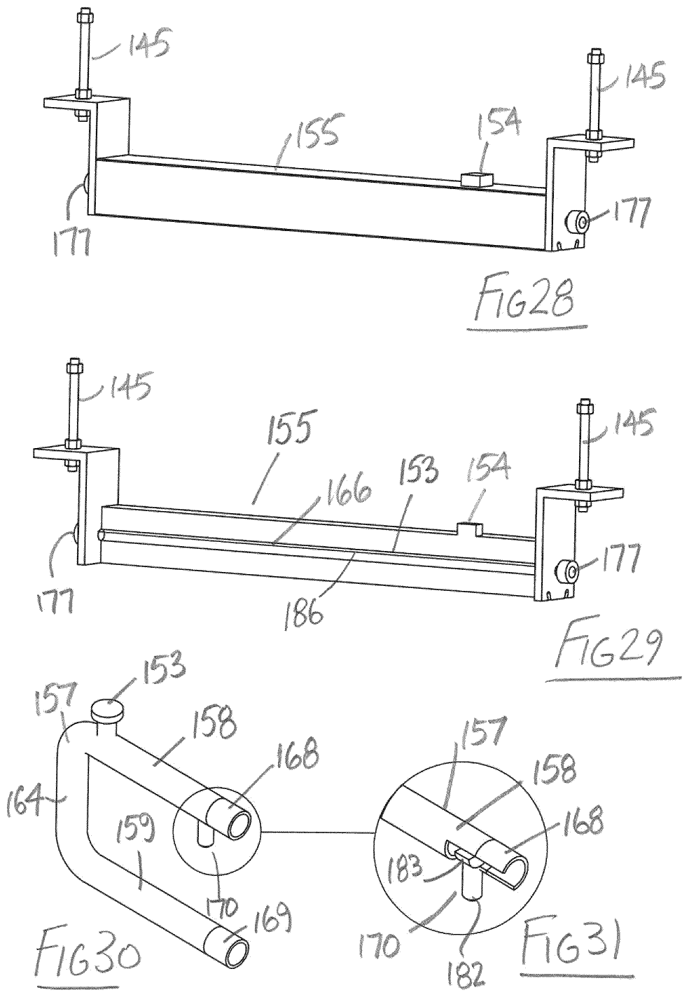

In this case the reservoir 135 comprises a number of stacks 148 of feeding troughs 156 which are spaced-apart and offset as best seen in FIG. 18. At a top of each stack 148 a sterilization light connector 154 is mounted on a header support tube 155 below which a plurality of feeding troughs 156 are stacked and interconnected at their ends by C-link pipes 157. Each sterilization light connector 154 is connected to a UV sterilization light 153 (FIG. 29) mounted within the header support tube 155 which is operable to sanitize liquid food passing through the header support tube 155.

Each feeding trough 156 has angled side walls 160 forming a landing and crawl area for wasps. A liquid food pool recess 161 forming a feeding channel extends along each side wall 160 into which a mesh insert 165 sits. The mesh insert 165 prevents debris from becoming lodged in the liquid food pool recess 161. An access opening 198 of each liquid food pool recess 161 is sufficiently narrow to prevent a wasp falling into the liquid food pool recess 161 whilst at the same time allowing the wasp to feed at the liquid food pool recess 161.

Male connector arms 162 project outwardly at a top of the feeding trough 156 and complementary female receiver slots 163 for reception of the male connector arms 162 of another feeding trough 156 are provided at a bottom of the feeding trough 156. Thus, the feeding troughs 156 slidably inter-engage for assembly into the stack 148 of a desired size, depending upon how many of the feeding troughs 156 are used. Each receiver slot 163 is open at one end at insert plate 181 to receive the connector arm 162. A stop plate 189 at an opposite end of the receiver slot 163 correctly aligns inter-engaged feeding troughs 156 and only allows insertion and removal from one end, that is the insert plate 181 end.

Vertically adjacent feeding troughs 156 are inter-connected by the C-link pipes 157. Each C-link pipe 157 has a horizontal top arm 158 and a bottom arm 159 which is substantially parallel to the top arm 158. A cross piece 164 interconnects outer ends of the top arm 158 and the bottom arm 159. Inner ends 168, 169 of the top arm 158 and the bottom arm 159 are inserted into associated inlet/outlet sockets 177 at each end of an internal pipeline 166 extending between opposite ends of each feeding trough 156.

An adjustable flow reducer 170 is mounted on each top arm 158. The flow reducer 170 has an adjusting screw 182 connected to a weir head 183 mounted within the top arm 158 which can be raised or lowered within the top arm 158 by means of the adjusting screw to adjust the liquid food level 186 in the top arm 158 and hence via the internal pipeline 166 and irrigation outlet pipes 167 within the associated liquid food pool recesses 161 of the associated feeding trough 156. Excess liquid flows over the weir head 183 and through the C-link pipe 157 and into the next feeding trough 156 below. A breather valve 153 is provided on the top arm 158.

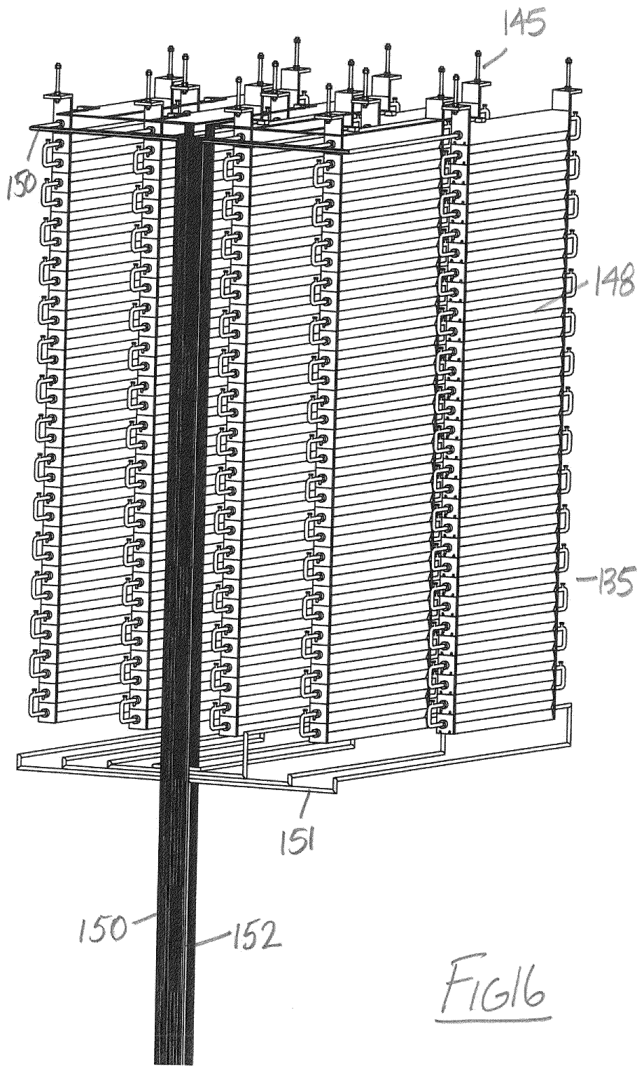

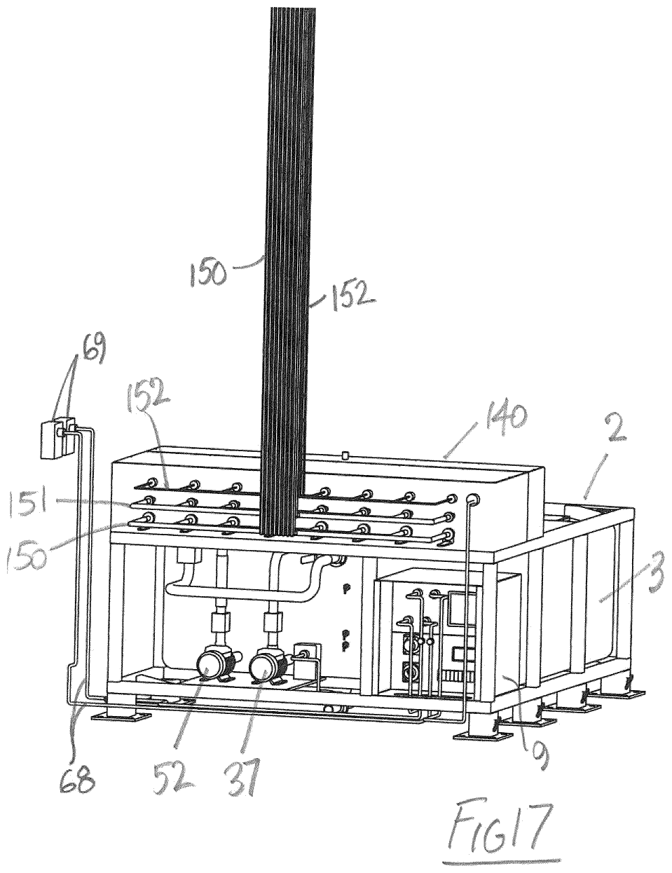

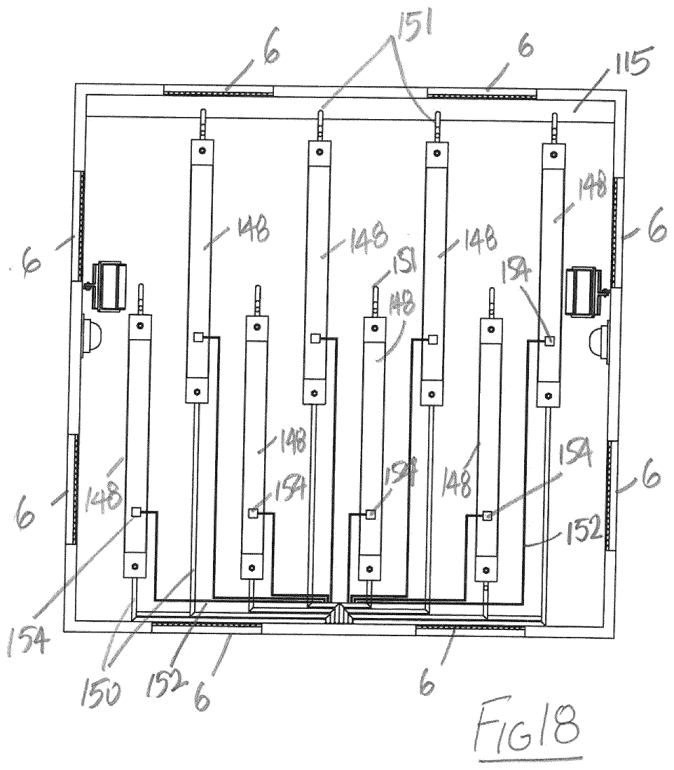

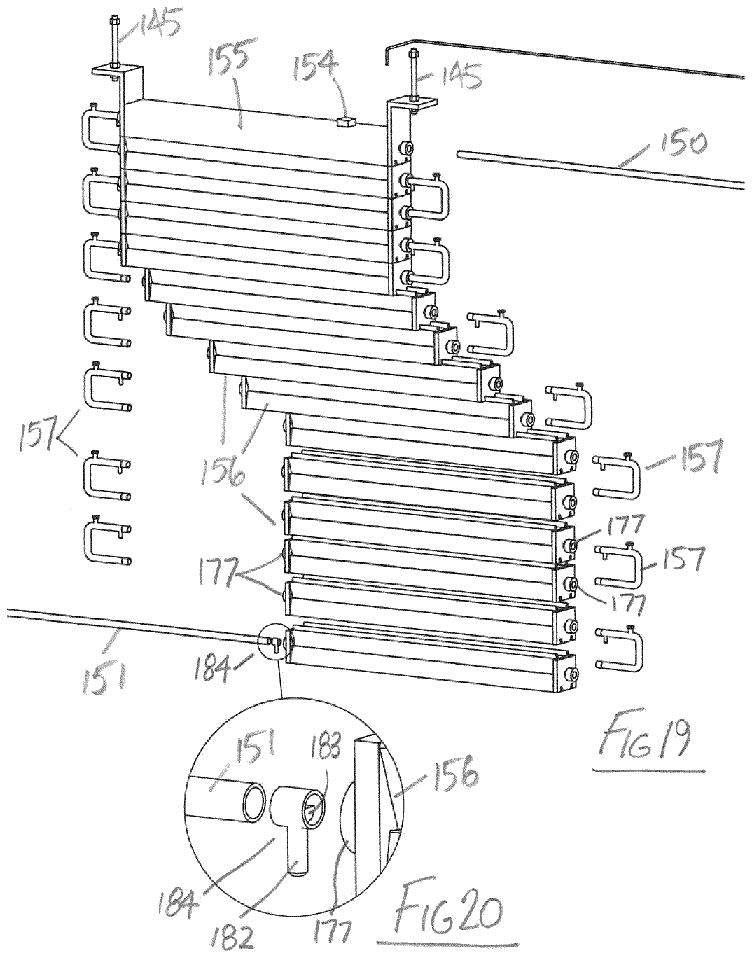

Liquid food is delivered from the liquid distribution manifold 140 by flow pipework 150 through the header support tube 155 and down through each feeding trough 156 in turn, filling the liquid food pool recesses 161 and discharging through return pipework 151 from the lowermost feeding trough 156 back to the liquid distribution manifold 140. A final reducer 184 (FIG. 19 and FIG. 20) is mounted at an outlet socket 177 of the lowermost feeding trough 156.

Each feeding trough 156 has an internal pipeline 166 with outwardly extending irrigation outlet pipes 167 which discharge liquid food into the liquid food pool recesses 161 at opposite sides of the feeding trough 156.

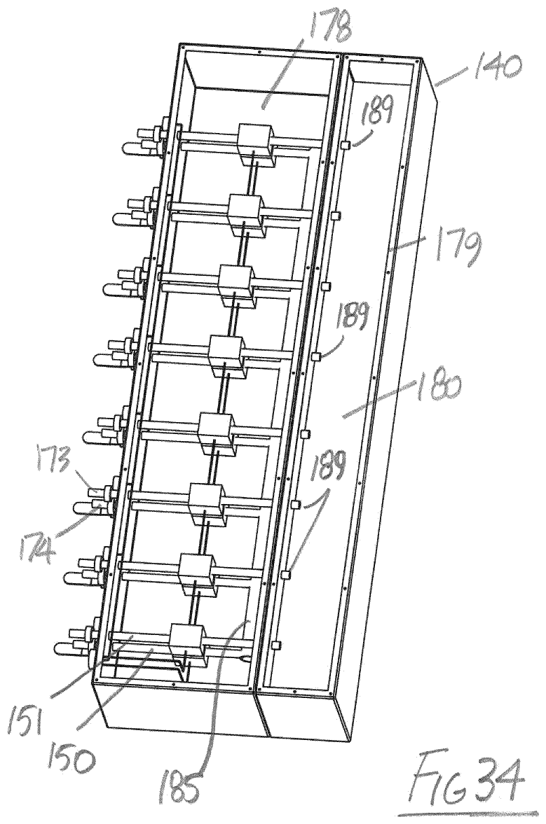

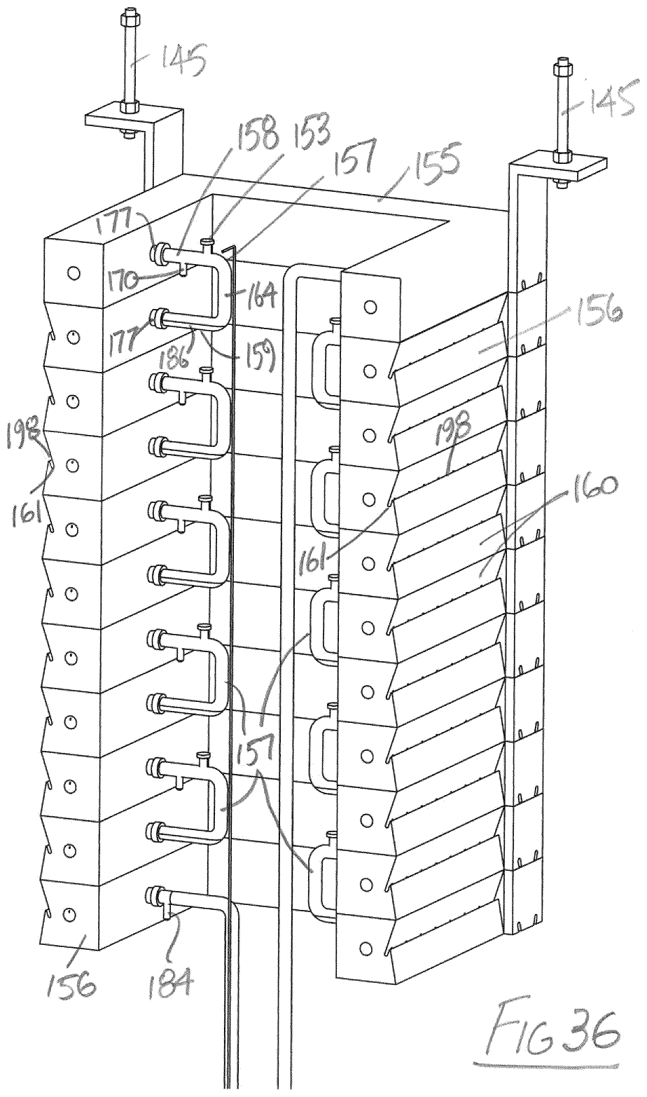

Referring in particular to FIG. 32 to FIG. 34, the liquid distribution manifold 140 has a feed chamber 178 and a return chamber 179. A filter 180 is mounted within the return chamber 179. Flow valves 171 connect to the flow pipework 150 and return valves 172 connect to the return pipework 151. Liquid food discharged from the feeder pump 52 is delivered via a flexible hose connector 176 to a feed rail 185 in the feed chamber 178 to which the flow valves 171 are connected. The return valves 172 discharge through discharge pipes 189 into a top of the return chamber 179 and liquid food passes through the filter 180 and is delivered by a flexible hose connector 176 to the main inlet valve 45 of the liquid food storage tank 3

Inline UV sterilization lights 187 in the flow pipework 150 and the return pipework 151 are contained within the feed chamber 178 of the liquid distribution manifold 140. Mounted on a faceplate 188 of the liquid distribution manifold 140 are connectors 173 to power the heater elements 154 on the header support tubes 155 via the connect cables 152. A multipin connector 192 connects the liquid distribution manifold 140 to the multiprocessor 63 via interconnect cables 68.

In use, as with the previously described embodiment a constant supply of liquid food is provided at the reservoir 135. The feeder pump 52 delivers liquid food through the feed rail 185 in the liquid distribution manifold 140 and then via the flow pipework 150 to each stack 148 of feeding troughs 156, entering at the header support tube 155 and percolating downwardly through each feeding trough 156 in turn. A constant liquid level 186 is maintained in each feeding trough 156 by means of the adjustable reducer 170 at an outlet of each feeding trough 156, excess liquid food being delivered downwardly by the C-link pipe 157 to the next below feeding trough 156 to maintain the liquid level 186 therein, and so on down through the whole stack 148 of feeding troughs 156. Excess liquid food is discharged from the lowermost feeding trough 156 through the final reducer 184, being delivered via the return pipework 151 to the return chamber 179 of the liquid distribution manifold 140. Liquid food then passes through the filter 180 to remove unwanted debris and impurities before return to the liquid food storage tank 3. Operation of the UV sterilization lights 153 on the header support tubes 155 at an inlet to each stack 148 sanitizes the liquid food.

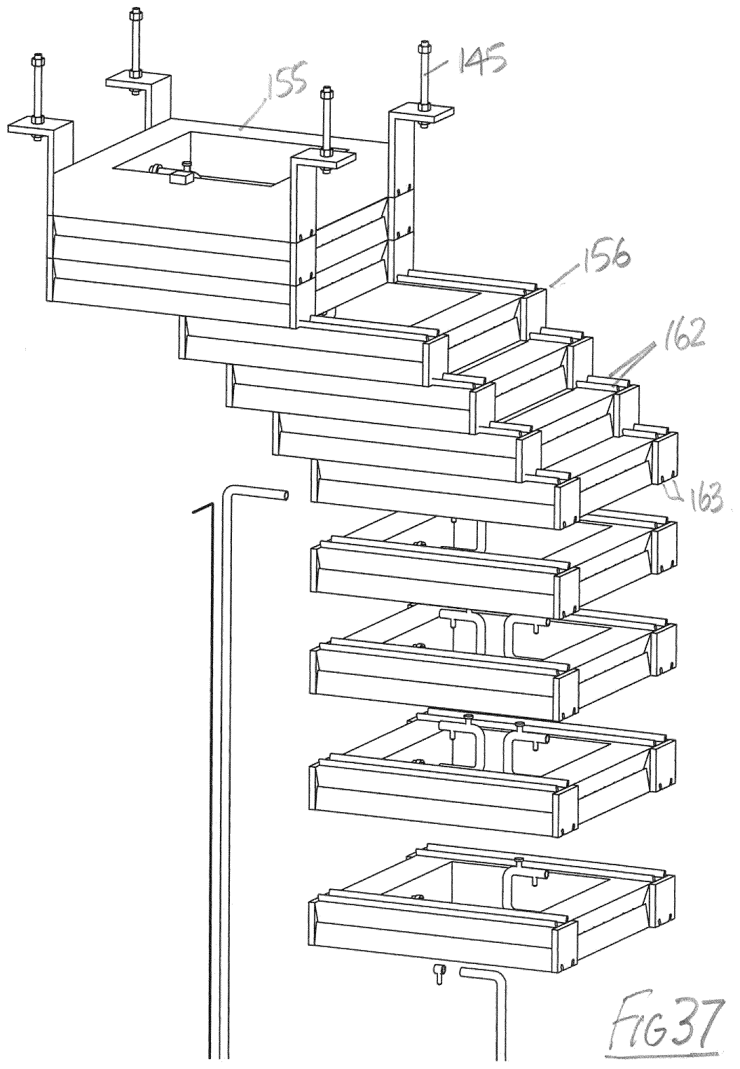

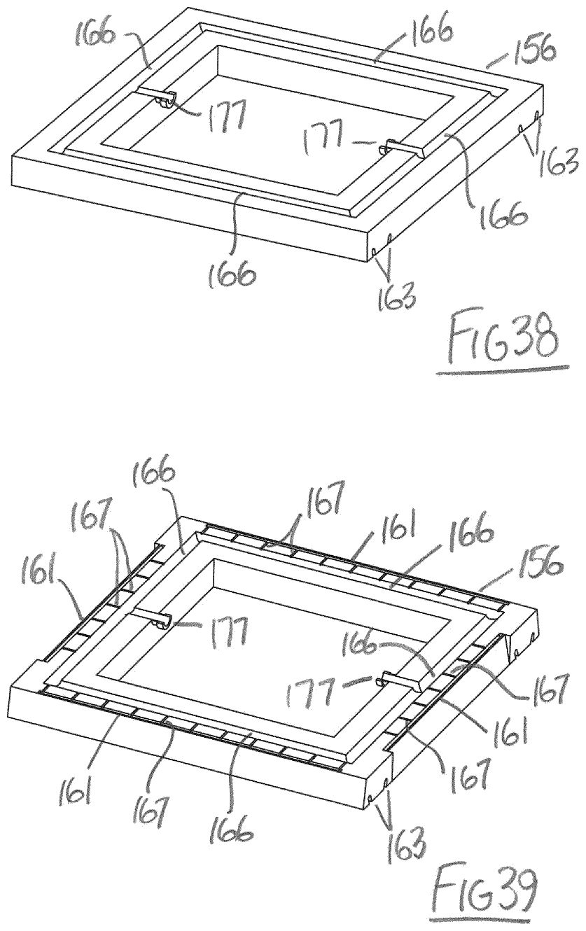

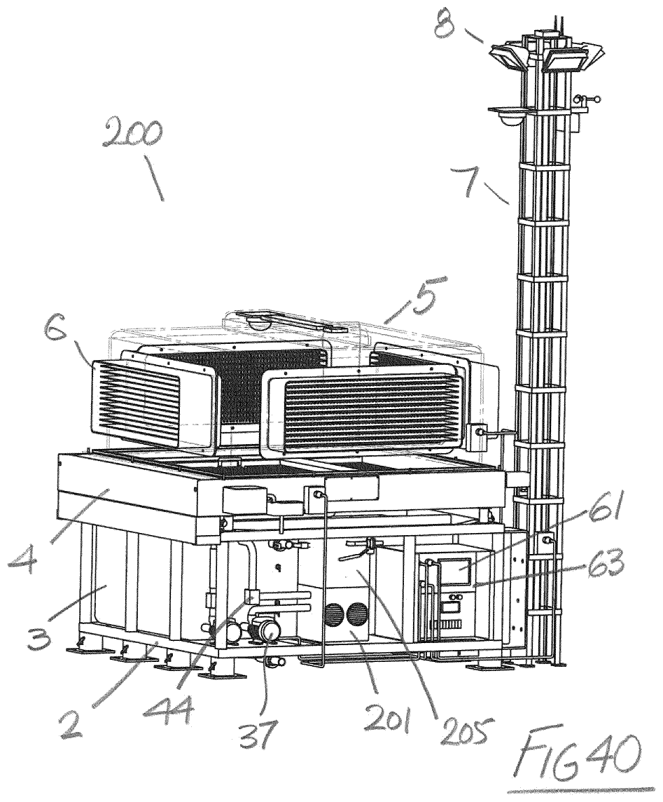

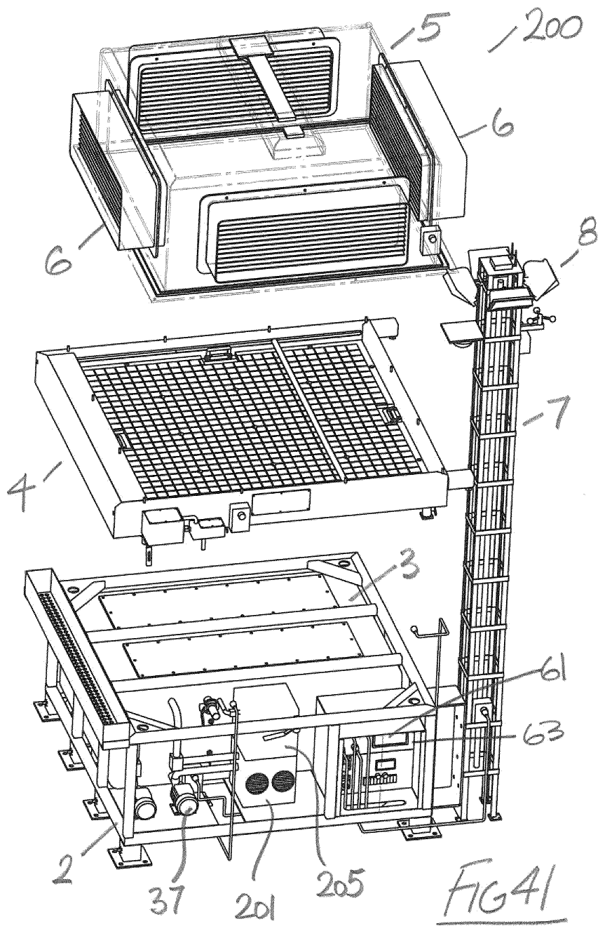

Referring now to FIG. 35 to FIG. 39, there is shown an alternative reservoir construction for another wasp feeding station according to a third embodiment of the invention. Parts similar to those described previously are assigned the same reference numerals. In this case the feeding troughs 156 are arranged in a ring formation with feeding channels at the exterior face and flow pipework situated at the rear face.

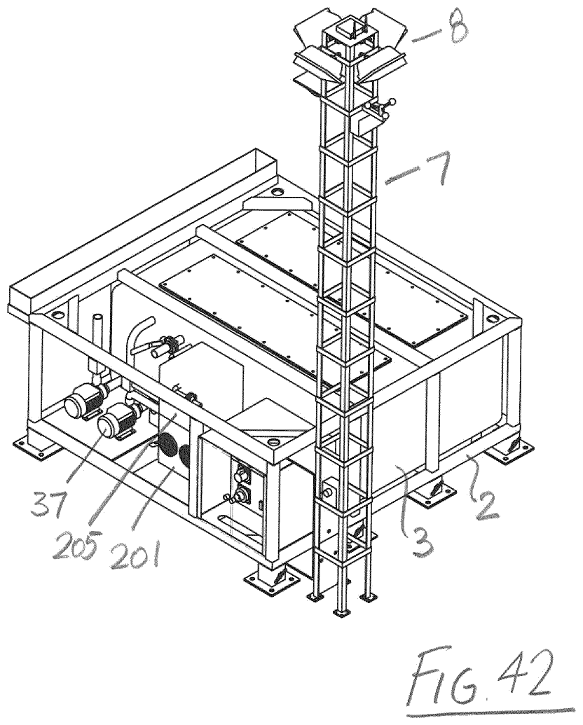

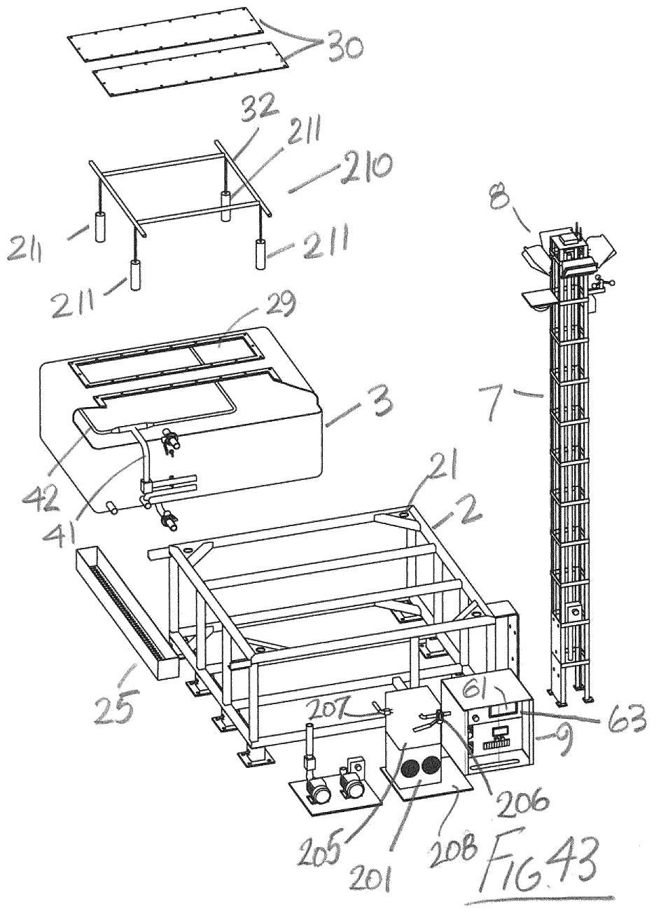

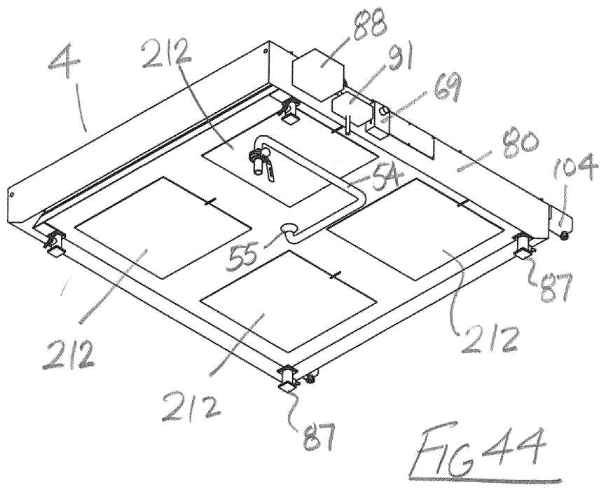

Referring now to FIG. 40 to FIG. 44, there is shown another wasp feeding station according to a further embodiment of the invention, indicated generally by the reference numeral 200, Parts similar to those described previously are assigned the same reference numerals.

In this case, the wasp feeding station 200 has a temperature control system 201 for regulating the temperature of the liquid food. The temperature control system 201 has a heating apparatus and a cooling apparatus for maintaining the liquid food within a desired temperature range. The mixer pump 37 draws liquid food from the liquid food storage tank 3 and delivers it through the temperature control system 201 for heating or cooling as required, before discharge back into the liquid food storage tank 3. Thus, the liquid food can be maintained within a desired temperature range.

Also mounted at a side wall of the liquid food storage tank 3 is an additive dosing tank 205 which is connected to the liquid food storage tank 3 for discharging additives into the liquid food stored therein. While the additives could be gravity fed into the liquid food storage tank 3, more preferably a dosing pump (not shown) is provided to deliver the required amount of additives into the liquid food storage tank 3. The dosing pump may conveniently be mounted within the additive dosing tank 205. The additive dosing tank 205 has an inlet valve 206. An outlet pipe 207 is connected from the additive dosing tank 205 to the liquid food storage tank 3. The temperature control system 201 and additive dosing tank 205 are mounted on a mounting plate 208 on the base frame 2.

In this case also, a sanitiser 210 is provided in the liquid food storage tank 3. The sanitiser 210 comprises a number of UV light emitting elements 211 suspended from the carrier frame 32 within the liquid food storage tank 3.

Thermal control pads 212 are also provided on an underside of the reservoir 4 and are operable to heat or cool liquid food within the reservoir 4.

The invention is an innovative solution that aims to solve the problems caused by the increasing numbers of scavenging wasps and the issues associated with their interaction with the public, when they descend upon inhabited areas, searching out new food sources in the summer and autumn months.

The wasp feeding station does not restrict the movement of wasps in anyway, housings protect the feeding zones from weather and debris while access ports allow for bidirectional free flow of wasps to and from the food source. The system provides wasps with a target specific nutrient rich non-toxic food that replaces their diminishing natural food supply. Feeding wasps for the duration of wasp pest season the units and system produce a method of ‘controlled relocation’ of wasp populations. In context, the wasp feeding station food source is neither a lure nor bait but simply a continuous supply.

The wasp feeding station is a non-kill-non-trap-non-toxic wasp pest control unit and system developed to be active in summer and autumn to resolve the problems and hazards associated with annual wasp pest infestations. Weatherproof, fully automated, secure and remotely monitored, the wasp feeding station is designed for permanent or seasonal deployment into elevated or on ground-based locations that are isolated from human interaction or intrusion.

Pioneering the method of a ‘controlled relocation’ of wasps by feeding, a coordinated network of active wasp feeding stations will produce wasp pest free areas in urban, rural, agricultural or industrial locations of any size or terrain with any scale of hive density or wasp population.

The system produces a method of ‘controlled relocation’ by supplying wasps with an optimum target specific, non-ceasing, liquid carbohydrate food. This nutritional syrup blend is irresistible for wasps and once discovered, wasps permanently abandon and discard all habitual wasp pest foraging areas and crops in favour of feeding exclusively from these dedicated oases.

Weatherproof housings contain access ports allowing fora continuous bidirectional free flow of wasps to and from the feeding zones. The system employs liquid flow methods that distribute liquid food into pooling canals and recesses contained on landing and crawl surfaces that are specifically engineered to facilitate effortless and continuous consumption of the food by wasps.

Engineered dispensary surfaces comprising of, dry landing and crawl areas, irrigated outlets and food pooling recesses and canals are purposely designed to enable continuous quantities of wasps to safely land and consume the liquid food with ease and efficiency and depart again with no risk of drowning or becoming trapped.

The wasp feeding station automatically delivers a constant and nutrient consistent supply of syrup for wasps to consume. The liquid food is specially blended and scented to be more desirable and convenient for wasps to eat than any other available fruit or manmade food sources.

A key feature design of the wasp feeding station is the food pooling technique, both reservoir pool and reservoir tube systems maintain a maximum or brimming level of obtainable liquid food at all times, this feature informs wasps of a non-depleting or limitless and abundant food supply at each and every feeding visit.

Once activated the system warms the liquid food, maximizing airborne aroma which alerts wasps by scent, enabling them to swiftly discover the locations of these purpose-built feeding stations.

The system continuously feeds wasps a non-ceasing supply of nutritional syrup for the duration of wasp pest season, safely removing them from adversely affected areas and crops. The feeding programme continues, resulting in a permanent relocation of wasp pest populations until the frosts of winter brings closure to the season.

A key aspect of design and operation is that all mechanical and electronic functions are automated and monitored. Operating systems and software allow the wasp feeding station to be networked and remotely managed. The feeding function is fully autonomous and the system is self-cleaning. Operational system data of functions are transmitted to and monitored from a base control station. Aside from periodic maintenance, once a wasp feeding station is activated there is no requirement for onsite personnel during an operating season. Camera system and lighting provide remote 24 hr video surveillance at a station. An active weather station relays 24 hr data on local conditions from each location. The stations have GPS location systems and are wireless or wired network and communication ready. Surge and ac protection are integral. Two separate counting systems are proposed to count wasps and monitor populations, an IR counting gate configuration and a camera system with counting software.

The wasp feeding station can facilitate many other insects and species for the purposes of—controlled relocation, propagation, feeding, scientific research, monitoring, protection, restoration and integrated pest management programmes.

In this specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described which may be varied in both construction and detail within the scope of the appended claims.

1. A wasp feeding station comprising:
a liquid food reservoir;
a liquid food storage tank connected to the reservoir for delivery of liquid food from the liquid food storage tank to the reservoir;
the liquid food storage tank being connected to the liquid food reservoir by a liquid food supply system which is operable to deliver liquid food from the liquid food storage tank to the liquid food reservoir to maintain a constant supply of liquid food in the liquid food reservoir,
the liquid food reservoir comprising at least one feeding channel having an access opening sufficiently narrow to prevent a wasp falling into the feeding channel, and a landing pad is provided adjacent the access opening of the feeding channel
wherein the liquid food reservoir comprises a feeding pool having a base with upstanding side walls, a feeding plate mounted within the pool, the feeding plate having a plurality of feeding channels for reception of liquid food, and
wherein a scraper blade extends between opposite side walls of the feeding pool and engages a top surface of the feeding plate, the scraper blade having a drive assembly for movement of the scraper blade across the top surface of the feeding plate.

2. (canceled)

3. (canceled)

4. (canceled)

5. The wasp feeding station as claimed in claim 1, wherein the reservoir is mounted within a weather protection enclosure having at least one access port to allow through passage of wasps to feed at the reservoir and to exit the weather protection enclosure.

6. The wasp feeding station as claimed in claim 1, further including a temperature control system for regulating the temperature of the liquid food.

7. The wasp feeding station as claimed in claim 6, wherein the temperature control system has heating apparatus and/or cooling apparatus for maintaining the liquid food within a desired temperature range.

8. The wasp feeding station as claimed in claim 1, wherein a sterilizer is mounted within the liquid food storage tank for sanitising liquid food therein.

9. The wasp feeding station as claimed in claim 8, wherein the sterilizer comprises a UV light source.

10. The wasp feeding station as claimed in claim 1, wherein an additive dosing tank is connected to the liquid food storage tank for delivery of additives into the liquid food.

11. (canceled)

12. (canceled)

13. The wasp feeding station as claimed in claim 1, wherein one or more thermal pads are mounted on the base of the reservoir, a thermostat associated with the thermal pads being operable to control operation of the thermal pads to maintain the temperature of liquid food in the reservoir within a desired temperature range by heating or cooling the liquid food in the reservoir.

14. (canceled)

15. The wasp feeding station as claimed in claim 1, wherein the reservoir has a level adjuster.

16. (canceled)

17. (canceled)

18. The wasp feeding station as claimed in claim 1, wherein the reservoir comprises a plurality of vertically stacked feed troughs.

19. The wasp feeding station as claimed in claim 18, wherein the feed troughs are connected by complementary interengagable formations on a top and on a bottom of each feeding trough.

20. The wasp feeding station according to claim 19, wherein the complementary interengagable formations comprise associated male and female connector elements.

21. The wasp feeding station as claimed in claim 1, wherein when the liquid food storage tank has a mixer for mixing liquid food stored in the storage tank.

22. The wasp feeding station as claimed in claim 21, wherein the mixer comprises a mixer pump mounted in a recirculating pipeline, the mixer pump having a mixer pump inlet connected to a storage tank outlet and a mixer pump outlet connected to a storage tank inlet.

23. The wasp feed station as claimed in claim 1, wherein a feed transfer pump has a feed transfer pump inlet connected to an outlet of the storage tank and a feed transfer pump outlet connected to an inlet of the reservoir.

24. The wasp feeding station as claimed in claim 5, wherein the weather protection enclosure has a plurality of spaced-apart landing pads mounted at each access port.

25. The wasp feeding station as claimed in claim 5, wherein an infra-red counting gate is mounted at each access port.

26. (canceled)

27. (canceled)

28. (canceled)

29. The wasp feeding station as claimed in claim 1, wherein the wasp feeding station includes monitoring and communication equipment to enable remote monitoring of the wasp feeding station.

30. (canceled)

31. The wasp feeding station as claimed in claim 29, wherein the monitoring and communication equipment includes one or more of cameras, lights, weather station, wireless connectivity and GPS.

32. The wasp feeding station as claimed in claim 18, wherein a filter is mounted in a liquid food return line communicating between the reservoir and the liquid food storage tank.

33. (canceled)
